(19) United States
(12) Patent Application Publication
(10) Pub. No.: US 2012/0195838 A1
(43) Pub. Date: Aug. 2, 2012

(54) ORAL COMPOSITION FOR DENTURE WEARERS

(75) Inventors: Yoshihito Naitou, Tokushima-shi (JP); Kenichi Hamada, Tokushima-shi (JP); Tetsuo Ichikawa, Tokushima-shi (JP); Yoichiro Miyake, Tokushima-shi (JP); Shoji Yahara, Kumamoto-shi (JP)

(73) Assignees: NATIONAL UNIVERSITY CORPORATION KUMAMOTO UNIVERSITY, Kumamoto (JP); THE UNIVERSITY OF TOKUSHIMA, Tokushima (JP)

(21) Appl. No.: 13/392,905

(22) PCT Filed: Sep. 2, 2010
(86) PCT No.: PCT/JP2010/064998

§ 371 Date: Apr. 5, 2012

(30) Foreign Application Priority Data
Sep. 4, 2009 (JP) 2009-204992

(51) Int. Cl. A61K 36/00; A61P 31/00

(52) U.S. Cl. 424/58

(57) ABSTRACT

Provided is an oral composition for denture wearers, which includes an antimicrobial substance of biological origin, is biocompatible, and has an antimicrobial activity. Specifically, provided is an antimicrobial oral composition for denture wearers including a component of common rush origin. The oral composition for denture wearers is placed in the mouth, and hence the oral composition for denture wearers including a component of common rush origin, which may also be used as a food additive, is safe for a living body and useful.

### TECHNICAL FIELD

The present invention relates to an oral composition for denture wearers, and more particularly, to an oral composition for denture wearers, which is subjected to antimicrobial treatment. The present invention also relates to a production method for an oral composition for denture wearers.

The present application claims priority of Japanese Patent Application No. 2009-204992, which is incorporated herein by reference.

### BACKGROUND ART

Progress of aging society in recent years cannot be overlooked any longer in dental medicine as well. As one becomes older, oral health enhances quality of life in the elderly, as is often said that good oral health is an indication of a healthy body and mind. Enhancement of masticatory performance through use of a denture or the like can lead to improvements in activities of daily living (ADL), vital functions (social roles), systemic health, and exercise performance, thereby enhancing life prognosis. The denture does not tend to decrease owing to an increase in elderly population.

The denture is placed in the mouth. It is therefore important to inhibit biofilm formation through an antimicrobial or microbicidal action to prevent opportunistic infections caused by oral indigenous bacteria and fungi and denture stomatitis. In order to keep the denture in a hygienic state, many denture cleansers are commercially available. In general, a peroxide-based denture cleanser does not have a strong harmful action, while it has a weak cleansing effect. A hypochlorous acid-based denture cleanser has a strong cleansing effect, while it is liable to damage a material for the denture as well. An enzyme-based denture cleanser has a mild cleansing effect, while it has less harmful property.

When the denture is placed in the mouth over a long period of time, an impression or a strain is generated in the oral mucosa, or a deformation is generated in the state of the alveolar ridge, resulting in a deterioration in fitting state and leading to insufficient adhesion of the denture. Thus, the denture may be liable to come off at the time of mouth opening or mastication. Usually, in such case, there is taken such a treatment that a relining material with soft quality is temporarily aligned on the mucosal side of the denture. A tissue conditioner is a material to be used in this treatment and is also called a denture temporary relining material. The tissue conditioner is required to have a low hardness and very soft performance. That is, with its softness, it is intended to not only recover a local deformation such as the impression or strain of the mucosa to an original normal state but also make the adhesion of the denture stable and improve the state of the alveolar ridge (Patent Literature 1). That is, the tissue conditioner is a lining material (relining material), which is brought into contact with the denture, and is widely used for adjustment of denture-supporting mucosa and the like.

However, it is considered that the tissue conditioner has a problem in that surface properties deteriorate through time-dependent outflow of components, and plaque formation occurs, resulting in increased risks of systemic influences on the elderly with weak resistance. In order to solve such problem, there is a report on a tissue conditioner including an inorganic component having antimicrobial property (Patent Literature 2). However, although a metal ion such as silver is stable, the denture is placed in the mouth, and it therefore cannot be denied that the denture may cause problems such as biocompatibility and development of metal allergy, and the like. Further, there is also a problem in that silver is one of the noble metals and hence is high in cost. In addition, the tissue conditioner deteriorates with time, and the antimicrobial component may also dissolve. In such case, it is desired that the antimicrobial component have particularly high safety for a living body.

In view of the foregoing, an attempt has been made at developing a tissue conditioner using an antimicrobial substance of biological origin. For example, there is a report on research for antimicrobial property of bamboo extract and its application in clinical dentistry (Non Patent Literature 1). However, the antimicrobial substance of biological origin includes various compounds, and it cannot be said that the compounds have been sufficiently elucidated for their problems of safety and the like. In actuality, a tissue conditioner including bamboo extract has not yet put into practical use.

Further, in the case of using the denture, a denture stabilizer is often used. The stabilizer is classified into three types, i.e., a powder type, a cream type, and a cushion type based on properties thereof. From the viewpoints of components and effects, the stabilizer is classified into a denture adhesive as a powder- or cream-type stabilizer including a highly adhesive component as a main component, and a home reliner as a cushioned rubbery stabilizer. The denture adhesive improves retention of the denture by allowing the denture adhere to the oral mucosa, and the home reliner improves retention of the denture by filling a gap between the denture and the oral mucosa to bring them in close contact with each other. The denture stabilizer is also applied to the mouth, and hence it is necessary to take care of sanitation, as is the case with the denture. There are some reports on a denture stabilizer composition having antimicrobial property. As a denture stabilizer composition, which has an excellent effect of suppressing the growth of Candida on a denture and has high safety, there is disclosed a denture stabilizer composition which has an excellent effect of suppressing the growth of Candida and besides is effective for prevention of candidiasis and prevention of Candida-induced denture stomatitis and oral malodor, obtained by blending polylysine and/or a salt thereof as a substance having high safety (Patent Literature 3). In addition, as a denture stabilizer, which has sustained germicidal properties on pathogens of oral diseases such as denture stomatitis, oral malodor, and periodontitis during a denture-wearing period, has an excellent effect of suppressing plaque adhererence in the mouth and on a denture base and the remaining teeth, and is satisfactory in usability and safety, there is disclosed a denture stabilizer obtained by blending a denture stabilizer containing a vinyl acetate resin and ethanol with tea tree oil or Eucalyptus globulus oil (Patent Literature 4).

There is a demand for development of an oral composition for denture wearers, which includes an antimicrobial substance of biological origin, is biocompatible, and has an antimicrobial activity.

### CITATION LIST

### Patent Literature

[PTL 1] JP 2000-245748 A

[PTL 2] JP 09-322906 A

[PTL 3] JP 2004-147961 A

[PTL 4] JP 2007-175090 A

### Non Patent Literature

[NPL 1] The Journal of the Japanese Society for Dental Materials and Devices, Vol. 15, No. 2, 146-149 (1996)

### SUMMARY OF INVENTION

### Technical Problem

An object of the present invention is to provide an oral composition for denture wearers, which includes an antimicrobial substance of biological origin, is biocompatible, and has an antimicrobial activity.

### Solution to Problem

The inventors of the present invention have paid their attention on a substance having an antimicrobial activity of biological origin and made intensive studies to achieve the object. As a result, the inventors have found that a component of common rush origin is miscible with a material component for an oral composition for denture wearers and has an excellent antimicrobial activity. Thus, the present invention has been completed. Further, the oral composition for denture wearers is placed to the mouth. Hence, the oral composition for denture wearers including a component of common rush origin, which may also be used as a food additive, is safe for a living body and useful. In view of the foregoing, the present invention has been completed through the use of the antimicrobial oral composition for denture wearers including a component of common rush origin.

That is, the present invention includes the following items.

1. An antimicrobial oral composition for denture wearers, including a component of common rush origin.
2. The antimicrobial oral composition for denture wearers according to the above-mentioned item 1, in which the component of common rush origin includes a powdery component obtained by drying and pulverizing common rush.
3. The antimicrobial oral composition for denture wearers according to the above-mentioned item 1 or 2, in which the oral composition for denture wearers includes any one selected from a group consisting of: a tissue conditioner; a denture stabilizer; a denture coating agent; a denture cleaning paste; a wound-dressing material for denture wearers after oral surgery; a sealing cement for denture wearers; a quick cure resin for denture wearers; a gum for denture wearers; and a troche for denture wearers.
4. The antimicrobial oral composition for denture wearers according to the above-mentioned item 3, in which the oral composition for denture wearers includes one of a tissue conditioner and a denture stabilizer.
5. The antimicrobial oral composition for denture wearers according to the above-mentioned item 4, in which the content of the component of common rush origin is 2.5 to 20% by weight with respect to a resin composition for one of the tissue conditioner and for the denture stabilizer.
6. A raw material for producing an antimicrobial oral composition for denture wearers, including: a component of common rush origin; and a resin composition for one of a tissue conditioner and for a denture stabilizer.
7. A production method for an antimicrobial oral composition for denture wearers, including mixing a component of common rush origin into a resin composition.
8. The production method for an antimicrobial oral composition for denture wearers according to the above-mentioned item 7, in which the component of common rush origin includes a powdery component obtained by drying and pulverizing common rush.
9. The production method for an antimicrobial oral composition for denture wearers according to the above-mentioned item 8, in which the mixing of the component of common rush origin is carried out at 2.5 to 20% by weight with respect to the resin composition.
10. The production method for an antimicrobial oral composition for denture wearers according to any one of the above-mentioned items 7 to 9, in which the antimicrobial oral composition for denture wearers includes one of a tissue conditioner and a denture stabilizer.

### Advantageous Effects of Invention

The antimicrobial oral composition for denture wearers of the present invention showed excellent results as compared to a control in Candida albicans. In addition, the antimicrobial oral composition for denture wearers exhibited a slight antimicrobial activity on Porphyromonas gingivalis as well. Further, among the antimicrobial oral compositions for denture wearers, the tissue conditioner showed results within the range of reference values in any of a consistency test, a penetration test, and a penetration ratio test, and satisfied mechanical properties necessary for the tissue conditioner. In addition, the antimicrobial oral composition for denture wearers of the present invention was found to have an excellent odor removal effect as well. Accordingly, the antimicrobial oral composition for denture wearers of the present invention includes a component of common rush origin, which may also be used as a food additive, and it is therefore possible to provide the antimicrobial oral composition for denture wearers, which is excellent in safety, and has a high antimicrobial activity.

In addition, the antimicrobial oral composition for denture wearers of the present invention may be produced with a resin composition for producing an oral composition for denture wearers into which a component of common rush origin has been incorporated in advance, or may also be produced by mixing a component of common rush origin into a resin composition for producing an oral composition for denture wearers before use.

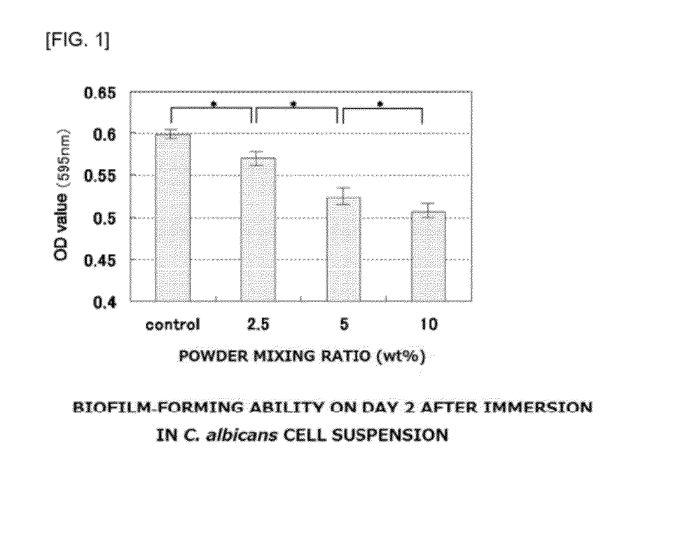

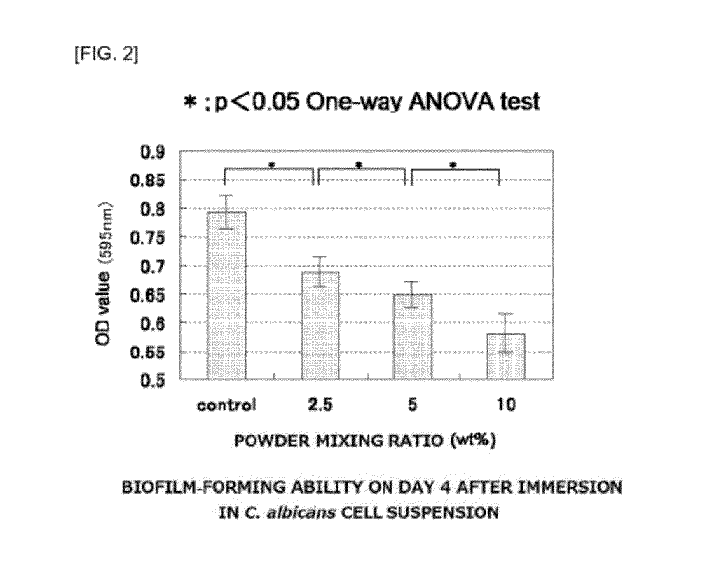

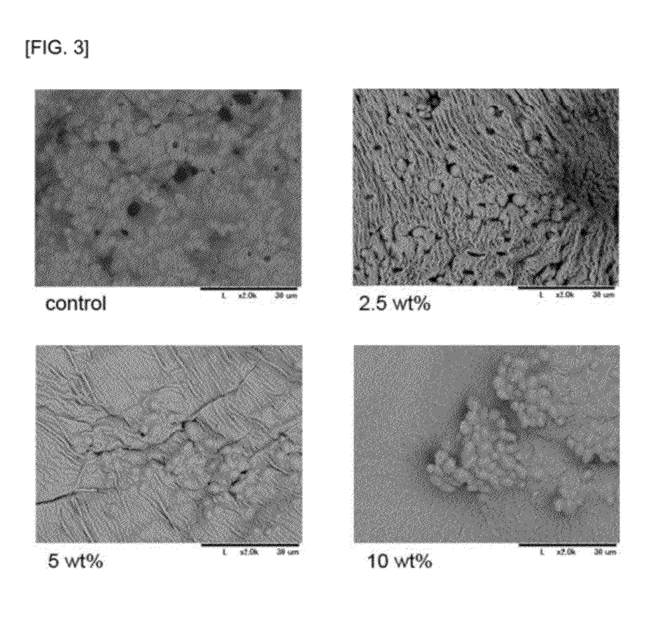

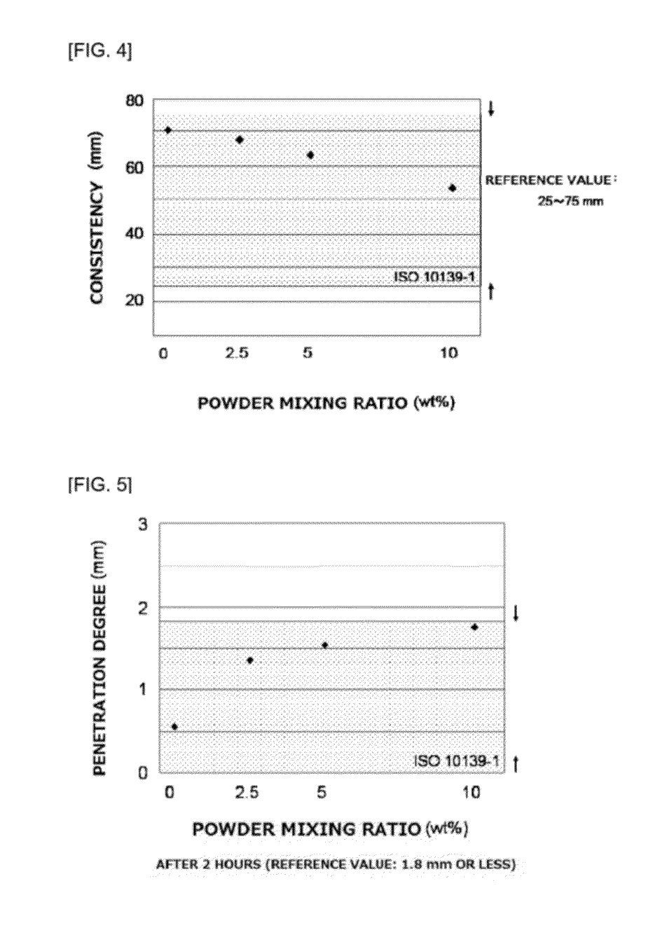

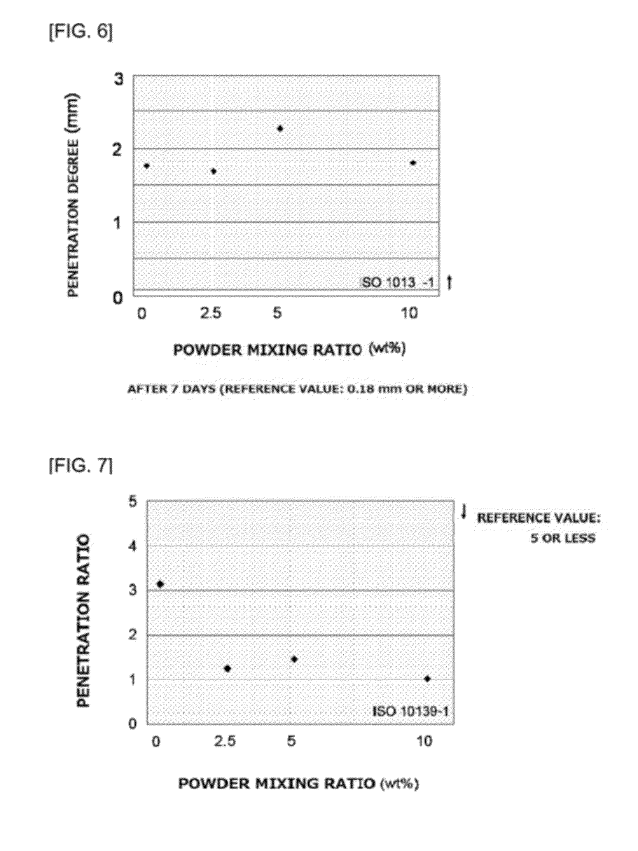

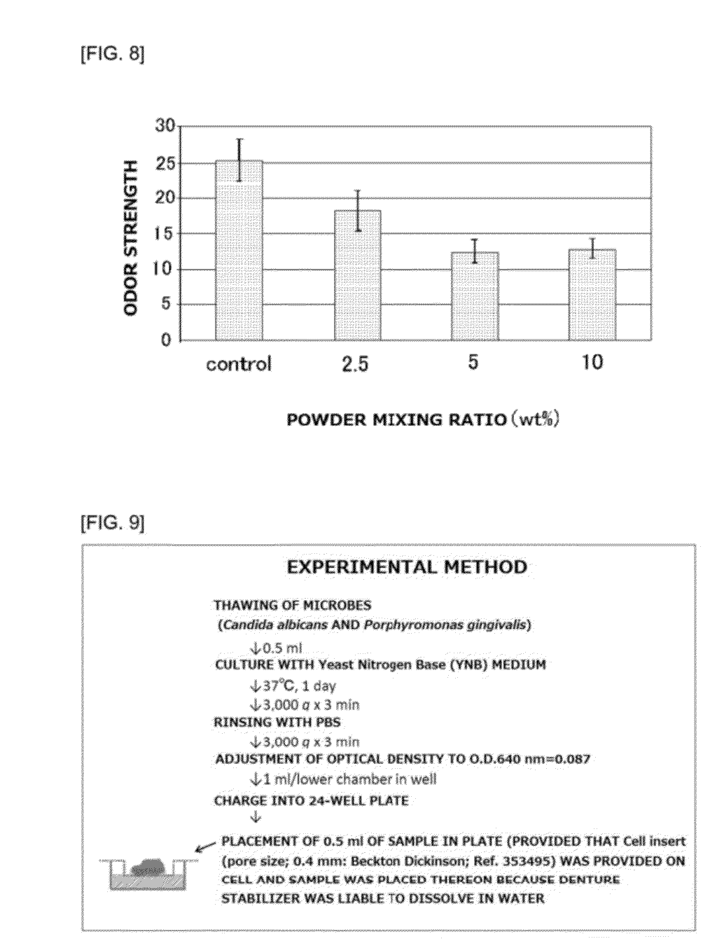

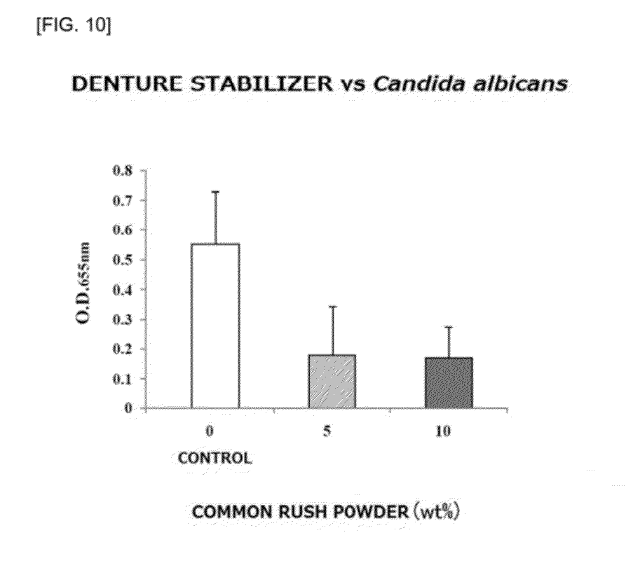

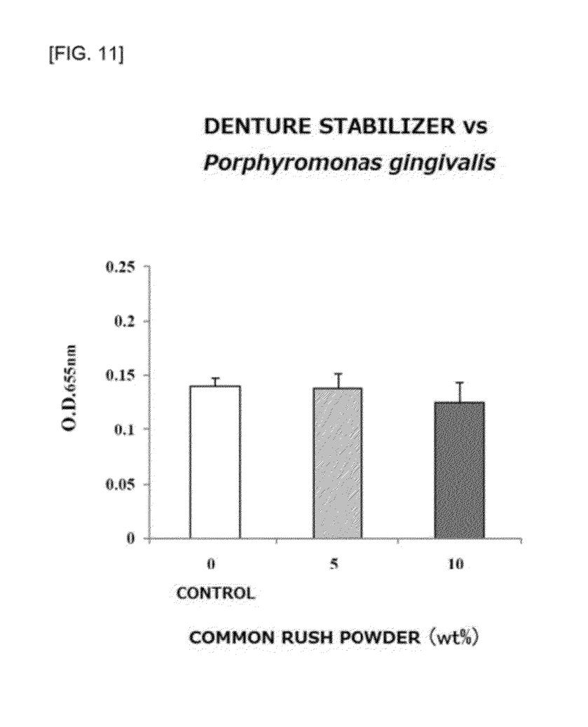

### BRIEF DESCRIPTION OF DRAWINGS

[FIG. 1] FIG. 1 is a graph showing biofilm-forming abilities on Day 2 of antimicrobial tissue conditioner specimens including a common rush powder mixed at different concentrations (Experimental Example 1-1).

[FIG. 2] FIG. 2 is a graph showing biofilm-forming abilities on Day 4 of antimicrobial tissue conditioner specimens including a common rush powder mixed at different concentrations (Experimental Example 1-1).

[FIG. 3] FIG. 3 is photographs showing the morphology of Candida albicans on surfaces of antimicrobial tissue conditioner specimens including a common rush powder mixed at different concentrations (Experimental Example 1).

[FIG. 4] FIG. 4 is a graph showing consistencies of antimicrobial tissue conditioner specimens including a common rush powder mixed at different concentrations (Experimental Example 1-2).

[FIG. 5] FIG. 5 is a graph showing penetration degrees after 2 hours of antimicrobial tissue conditioner specimens including a common rush powder mixed at different concentrations (Experimental Example 1-3).

[FIG. 6] FIG. 6 is a graph showing penetration degrees after 7 days of antimicrobial tissue conditioner specimens including a common rush powder mixed at different concentrations (Experimental Example 1-3).

[FIG. 7] FIG. 7 is a graph showing penetration ratios after 2 hours and after 7 days of antimicrobial tissue conditioner specimens including a common rush powder mixed at different concentrations (Experimental Example 1-3).

[FIG. 8] FIG. 8 is a graph showing observation results of odor removal effects of antimicrobial tissue conditioner specimens including a common rush powder mixed at different concentrations (Experimental Example 1-4).

[FIG. 9] FIG. 9 is a diagram illustrating an experimental method for measurement of antimicrobial activities of antimicrobial denture stabilizers including a common rush powder mixed at different concentrations on Candida albicans and Porphyromonas gingivalis (Experimental Example 2).

[FIG. 10] FIG. 10 is a graph showing antimicrobial activities of antimicrobial denture stabilizers including a common rush powder mixed at different concentrations on C. albicans (Experimental Example 2).

[FIG. 11] FIG. 11 is a graph showing antimicrobial activities of antimicrobial denture stabilizers including a common rush powder mixed at different concentrations on P. gingivalis (Experimental Example 2).

### DESCRIPTION OF EMBODIMENTS

The present invention relates to an antimicrobial oral composition for denture wearers, including a component of common rush origin. In the present invention, the oral composition for denture wearers has only to be used in the mouth by denture wearers, and is not particularly limited. Further, in this description, the denture may be a complete denture or a partial denture. Specific examples of the oral composition for denture wearers include a tissue conditioner, a denture stabilizer, a denture coating agent, a denture cleaning paste, a wound-dressing material for denture wearers after oral surgery, a sealing cement for denture wearers, a quick cure resin for denture wearers, a gum for denture wearers, a troche for denture wearers, artificial saliva for denture wearers, a denture cleanser, and a mouthwash for denture wearers. The oral composition for denture wearers suitably includes a resin composition.

In this description, the resin composition has only to be a resin which may be used in the mouth, and may be a resin known per se or a resin to be developed in the future. Specifically, the resin composition may be a natural resin or a resin produced by synthesis. A resin to be used for the tissue conditioner may be, for example, a commercially available tissue conditioner resin composition. Specific examples thereof include a Soft-Liner™ (manufactured by GC DENTAL PRODUCTS CORP.) and a SHOFU™ Tissue-Conditioner (manufactured by SHOFU INC.). Further, there may also be used a temporary relining material resin composition described in Patent Literature 1. Further, a resin to be used for the denture stabilizer may be exemplified by an alkyl vinyl ether-maleic anhydride copolymer, a salt thereof, and a derivative thereof, polyacrylic acid or a copolymer precursor of polyacrylic acid, a mixed Na/Ca salt of amethyl vinyl ether-maleic acid copolymer, a substantially anhydrous mixture of sodium carboxymethylcellulose and a trivalent cation, and other resins disclosed in Patent Literatures 3 and 4.

In this description, the antimicrobial oral composition for denture wearers includes a component of common rush origin. In this case, as the component of common rush origin, there may be used a powdery component obtained by drying and pulverizing harvested common rush. Alternatively, there may be used a component of common rush origin extracted by adding water to powdery common rush. The extent of pulverization of the common rush necessary for providing the powdery component is not particularly limited. However, the common rush is suitably pulverized to such an extent as to provide particles each having a size that allows the component of common rush origin to be uniformly dispersed in the antimicrobial oral composition for denture wearers in the mixing of the component of common rush origin. The powdery component of common rush origin may be added at 2.5 to 20% by weight, more preferably 2.5 to 10% by weight with respect to the antimicrobial oral composition for denture wearers of the present invention.

The component of common rush origin included in the antimicrobial oral composition for denture wearers of the present invention may be uniformly and easily mixed into the resin composition to be used in the production of the antimicrobial oral composition for denture wearers. Thus, an antimicrobial effect is exhibited in a uniform state. Meanwhile, in the case where the component of common rush origin is mixed with the resin composition to be used in the production of the antimicrobial oral composition for denture wearers, when there is fear in that unfavorable coloring or the like may occur in the oral composition for denture wearers, other components may further be incorporated in order to deal with such problem.

The antimicrobial oral composition for denture wearers of the present invention exhibits an antimicrobial activity in an antimicrobial test on each of C. albicans and P. gingivalis. Further, the antimicrobial oral composition is expected to have an antimicrobial activity on Streptococcus mutans as well. In particular, the antimicrobial oral composition for denture wearers of the present invention exhibits an excellent antimicrobial activity on C. albicans. C. albicans forms the so-called denture plaque, which causes an inflammation under a complete denture, a partial denture, or the like, i.e., denture stomatitis, and forms a biofilm (Jan. J. Mycol. Vol. 40, 233-242 (2005)). The antimicrobial oral composition for denture wearers of the present invention exhibits a particularly excellent antimicrobial activity on C. albicans, and hence is useful for denture wearers.

The present invention also encompasses a production method for an antimicrobial oral composition for denture wearers. For example, in the production of an antimicrobial oral composition for denture wearers, the antimicrobial oral composition for denture wearers may be produced by adding a component of common rush origin to a resin for an oral composition for denture wearers before use, or may be produced with a material including a component of common rush origin mixed in advance into a resin for an oral composition for denture wearers. The present invention also encompasses a raw material for producing an antimicrobial oral composition for denture wearers including a component of common rush origin mixed into a resin composition as a material for producing an antimicrobial oral composition for denture wearers.

When the antimicrobial oral composition for denture wearers of the present invention is specifically an antimicrobial tissue conditioner, the present invention also encompasses a production method for an antimicrobial tissue conditioner. For example, in the production of an antimicrobial tissue conditioner, the antimicrobial tissue conditioner may be produced by adding a component of common rush origin to a tissue conditioner resin composition before use, or may be produced with a material including a component of common rush origin mixed in advance into a tissue conditioner resin composition. The present invention also encompasses a raw material for producing an antimicrobial tissue conditioner including a component of common rush origin mixed into a resin composition as a material for producing a tissue conditioner.

### EXAMPLES

Hereinafter, the present invention is more specifically described by way of examples. However, the present invention is not limited to these examples.

### Example 1

### Production of Antimicrobial Tissue Conditioner

A commercially available tissue conditioner (manufactured by GC, Tokyo) was used as the resin composition for producing an antimicrobial tissue conditioner of the present invention, and a common rush powder (Inada, Yatsushiro) was used as the component of common rush origin. The resin composition was mixed at a powder-liquid ratio specified by the manufacturer. In addition, the common rush powder was added so as to be 0, 2.5, 5, and 10% by weight.

### Experimental Example 1-1

### Biofilm Formation Inhibition Test

The antimicrobial tissue conditioners including a common rush powder mixed at different concentrations produced in Example 1 were used to produce discoidal specimens each having a diameter 13 mm and a height of 2 mm. The specimens thus produced were sterilized with ultraviolet rays for 24 hours and then immersed in a C. albicans cell suspension at 1×10⁵ CFU/ml. After the immersion for 2 days and 4 days, the specimens were sufficiently washed with water, and biofilms on surfaces of the specimens were recovered. Then, the solutions were each measured for its optical density at 595 nm to evaluate a biofilm-forming ability of each of the specimens. Further, the morphology of Candida albicans on each of the specimens was observed.

The results revealed that a significant reduction in optical density was found in the specimens including a common rush powder at 2.5% by weight or more as compared to a control specimen including no common rush powder, suggesting that biofilm formation by Candida albicans was inhibited (FIGS. 1 and 2). The results also revealed that, for the morphology of Candida albicans on Day 4 after the immersion, higher shrinkage deformation was observed in the case where the common rush powder was mixed at a higher concentration, suggesting that the component of common rush origin had an antimicrobial effect (FIG. 3).

### Experimental Example 1-2

### Consistency Test

A consistency test is one of the measurement methods for the softness, fluidity, and the like of a material. The test is one of the evaluation methods for mechanical properties of a tissue conditioner and needs to meet the ISO10139-1 specification. In this test example, 2 ml of each sample of the antimicrobial tissue conditioners including a common rush powder mixed at different concentrations produced in Example 1 were weighed, mixed, and then placed on a glass sheet. Another glass sheet (100 g) was placed still on the sample 30 seconds after the completion of the mixing. A weight of 1,000 g was vertically placed still on the glass sheet 120 seconds after the completion of the mixing, and 1,100 g of a load were applied. The weight was removed 60 seconds later, and the spread sample was measured for its dimensions of the maximum and minimum parts between parallel tangents 8 minutes after the start of the mixing. The test was carried out four times, and an average of the resultant values was defined as a consistency.

Reference values for the consistency are 25 to 75 mm. As a result of the test, the consistency of each sample all showed a value within the range of the reference values (FIG. 4). The antimicrobial tissue conditioner of the present invention was found to be sufficient for clinical applications in terms of mechanical properties determined using the consistency as an indicator.

### Experimental Example 1-3

### Penetration Degree and Penetration Ratio Tests

A penetration degree test is one of the measurement methods for the hardness of a material. A needle having a predetermined weight is vertically penetrated into a sample, and the hardness of the sample is expressed by the penetration depth. For a penetration degree value, a penetration degree of 1 means 1/10 mm, and a material having a larger number is assessed to be a softer material. The test is one of the evaluation methods for the mechanical properties of a tissue conditioner and needs to meet the ISO10139-1 specification. In this test example, each sample of the antimicrobial tissue conditioners including a common rush powder mixed at different concentrations produced in Example 1 was placed in a mold having a diameter of 10 mm and a height of 15 mm to produce a columnar specimen for a penetration degree test. The specimen was taken out from the mold 10 minutes after the start of the mixing, a Vicat needle was brought into contact with a surface of the specimen 120 minutes later, and a penetration depth was measured with a dial gauge 1.5 seconds later. The same test was further carried out twice for other fresh portions in the specimen, and an average value of the tests of three times in total was determined. The value was defined as a penetration degree A. The specimen was placed in a thermostat water bath again at 37° C., and the test was repeated 7 days later, and the resultant value was defined as a penetration degree B. Calculation was carried out with the equation of σ=A/B (σ: penetration ratio, A: penetration degree after 120 minutes, B: penetration degree after 7 days) to determine a penetration degree.

Reference values for the penetration degree after 120 minutes are 1.8 mm or less, and reference values for the penetration degree after a lapse of 7 days are 0.18 mm or more. The penetration degree of each sample showed a value within the range of the reference values in any case (FIGS. 5 and 6). In addition, reference values for the penetration ratio are 5 or less, and the penetration ratio of each sample showed a value within the range of the reference values in any case (FIG. 7). The results revealed that the antimicrobial tissue conditioner of the present invention was sufficient for clinical applications in terms of mechanical properties determined using the penetration degree and the penetration ratio as indicators.

### Experimental Example 1-4

### Odor Removal Test

In this test example, each sample of the antimicrobial tissue conditioners including a common rush powder mixed at different concentrations of Example 1 was molded with a mold having a diameter of 10 mm and a height of 15 mm to produce a specimen for an odor removal test. The specimen was immersed in distilled water in a thermostat bath at 37° C. The specimen was taken out 10 days later and measured for its odor strength with a handheld odor meter OMX-SR (SHINYEI Technology Co., LTD.). Measurement was carried out five times for each condition.

An odor removal effect was observed in all of the specimens including a common rush powder mixed (FIG. 8). Further, in a sensory test in humans, 4 out of 5 subjects felt that “an odor was reduced” for each of the specimens with a common rush powder blended at 5% by weight or more.

### Example 2

### Production of Antimicrobial Denture Stabilizer

A commercially available denture stabilizer (manufactured by Earth Chemical Co , Ltd.) was used as a resin composition for producing the antimicrobial denture stabilizer of the present invention, and a common rush powder (Inada, Yatsushiro) was used as the component of common rush origin. The common rush powder was added so as to be 0, 5, and 10% by weight with respect to the denture stabilizer.

### Experimental Example 2

### Antimicrobial Activity Test

The antimicrobial denture stabilizers produced in Example 2 were each observed for its antimicrobial activities on C. albicans and P. gingivalis.

0.5 ml of the antimicrobial denture stabilizers was used as samples. The samples were subjected to sterilization treatment with ultraviolet rays for 24 hours before this experiment.

Various cryopreserved microbial strains were thawed and cultured at 37° C. for 1 day with a yeast nitrogen base (YNB) medium (manufactured by Sigma). The culture solutions were centrifuged at 3,000 g for 3 minutes, and the precipitates were washed with PBS, further centrifuged at 3,000 g for 3 minutes, and diluted with PBS to adjust an optical density (OD) at a wavelength of 640 nm to 0.087, to thereby prepare diluted microbial solutions. Each of the diluted microbial solutions was charged into a 24-well plate, each of the samples was added thereto, culture was carried out at 37° C. for 17 hours, and then each of the dilution microbial solutions was measured for its optical density (wavelength: 655 nm). It should be noted that a Cell Culture Insert (pore size: 0.4 mm; Becton Dickinson Labware) was provided in the plate and each of the samples was placed thereon because each of the antimicrobial denture stabilizers produced in Example 2 was liable to absorb water. A flowchart for the experimental method is illustrated in FIG. 9.

The results revealed that after the culture for 17 hours, each of the denture stabilizers including a common rush powder exhibited an excellent antimicrobial activity on C. albicans, and in the case where the content of the common rush component was 10% by weight, each of the denture stabilizers exhibited a slight antimicrobial activity on P. gingivalis as well (FIGS. 10 and 11).

### INDUSTRIAL APPLICABILITY

As described in detail above, the antimicrobial oral composition for denture wearers of the present invention showed excellent results as compared to a control in C. albicans. In addition, the antimicrobial oral composition for denture wearers exhibited a slight antimicrobial activity on P. gingivalis as well. Further, among the antimicrobial oral compositions for denture wearers, the tissue conditioner showed the results within the range of the reference values in any of the consistency test, the penetration test, and the penetration ratio test, and satisfied mechanical properties necessary for the tissue conditioner. In addition, the antimicrobial oral composition for denture wearers of the present invention was found to have an excellent odor removal effect as well. Accordingly, the antimicrobial oral composition for denture wearers of the present invention includes a component of common rush origin which may also be used as a food additive use, and it is therefore possible to provide an antimicrobial oral composition for denture wearers, which is excellent in safety, and has a high antimicrobial activity.

In addition, the antimicrobial oral composition for denture wearers of the present invention may be produced with an oral composition for denture wearers for producing a resin composition into which a component of common rush origin has been incorporated in advance, or may be produced by mixing a component of common rush origin into an oral composition for denture wearers for producing resin composition before use.

According to the antimicrobial oral composition for denture wearers of the present invention, it is possible to provide the oral composition for denture wearers having excellent antimicrobial property, specifically, a tissue conditioner, a denture stabilizer, a denture coating agent, a denture cleaning paste, a wound-dressing material for denture wearers after oral surgery, a sealing cement for denture wearers, a quick cure resin for denture wearers, a gum for denture wearers, a troche for denture wearers, artificial saliva for denture wearers, a denture cleanser, a mouthwash for denture wearers, and the like.

1. An antimicrobial oral composition for denture wearers, comprising a component of common rush origin.

2. The antimicrobial oral composition for denture wearers according to claim 1, wherein the component of common rush origin comprises a powdery component obtained by drying and pulverizing common rush.

3. The antimicrobial oral composition for denture wearers according to claim 1, wherein the oral composition for denture wearers comprises any one selected from a group consisting of: a tissue conditioner; a denture stabilizer;
a denture coating agent; a denture cleaning paste; a wound-dressing material for denture wearers after oral surgery; a sealing cement for denture wearers; a quick cure resin for denture wearers; a gum for denture wearers; and a troche for denture wearers.

4. The antimicrobial oral composition for denture wearers according to claim 3, wherein the oral composition for denture wearers comprises of a tissue conditioner or a denture stabilizer.

5. The antimicrobial oral composition for denture wearers according to claim 4, wherein the content of the component of common rush origin comprises at 2.5 to 20% by weight with respect to a resin composition for one of a tissue conditioner and for a denture stabilizer.

6. A raw material for producing an antimicrobial oral composition for denture wearers, comprising: a component of common rush origin; and a resin composition for one of a tissue conditioner and for a denture stabilizer.

7. A production method for an antimicrobial oral composition for denture wearers, comprising blending a resin composition with a component of common rush origin.

8. The production method for an antimicrobial oral composition for denture wearers according to claim 7, wherein the component of common rush origin comprises a powdery component obtained by drying and pulverizing common rush.

9. The production method for an antimicrobial oral composition for denture wearers according to claim 8, wherein the component of common rush origin is blended at 2.5 to 20% by weight with respect to a resin composition.

10. The production method for an antimicrobial oral composition for denture wearers according to claim 7, wherein the antimicrobial oral composition for denture wearers comprises one of a tissue conditioner and a denture stabilizer.
